(19) United States
(12) Patent Application Publication
(10) Pub. No.: US 2008/0103325 A1
(43) Pub. Date: May 1, 2008

(54) Mixed metal oxide catalysts for the ammoxidation of propane and isobutane

(76) Inventors: Claus Lugmair, San Jose, CA (US); Benjamin Mork, New Port Beach, CA (US); Jessica Zysk Fryer, San Francisco, CA (US); Anthony F. Volpe, Santa Clara, CA (US); Joseph Peter Bartek, Wheaton, IL (US); Hailian Li, Fremont, CA (US); Alakananda Bhattacharyya, Glen Ellyn, IL (US); James F. Brazdil, Glen Ellyn, IL (US); Bruce I. Rosen, Park Ridge, IL (US); Eric Moore, Wheaton, IL (US)

Correspondence Address: David P. Yusko, S/600, 4225 Naperville Road, Lisle, IL, 60532, US

(21) Appl. No.: 11/590,153

(22) Filed: Oct. 31, 2006

(51) Int. Cl. C07C 253/26

(52) U.S. Cl. 558/321; 502/302; 502/303

(57) ABSTRACT

A catalyst composition comprising molybdenum, vanadium, antimony niobium, at least one element select from the group consisting of titanium, tin, germanium, zirconium, and hafnium, and at least one lanthanide selected from the group consisting of lanthanum, praseodymium, neodymium, samarium, europium, gadolinium, terbium, dysprosium, holmium, erbium, thulium, ytterbium and lutetium; with the proviso that catalyst contains germanium (in the absence of at last one of titanium, tin, zirconium, hafnium) only in combination with neodymium and/or praseodymium and no other lanthanides. Such catalyst compositions are effective for the gas-phase conversion of propane to acrylonitrile and isobutane to methacrylonitrile (via ammoxidation).

### REFERENCE TO RELATED APPLICATIONS

This application is related to U.S. patent application Ser. No. ______, entitled “Lithium Containing Mixed Metal Oxide Catalysts for the Ammoxidation of Propane and Isobutane”, filed DATE by Rosen et al. under Attorney Docket No. 00087, filed on the date even herewith, which is hereby incorporated by reference for all purposes.

### BACKGROUND OF THE INVENTION

1. Field of the Invention

The present invention generally relates to catalyst compositions, methods of preparing such catalyst compositions, and methods of using such catalyst compositions for the gas-phase conversion of propane to acrylonitrile and isobutane to methacrylonitrile (via ammoxidation) or of propane to acrylic acid and isobutane to methacrylic acid (via oxidation).

The invention particularly relates to catalyst compositions, methods of preparing such catalyst compositions, and methods of using such catalyst compositions, where in each case, the same comprises molybdenum, vanadium, antimony, niobium, at least one element select from the group consisting of titanium, tin, zirconium, and hafnium, and at least one lanthanide selected from the group consisting of lanthanum, praseodymium, neodymium, samarium, europium, gadolinium, terbium, dysprosium, holmium, erbium, thulium, ytterbium and lutetium.

2. Description of the Prior Art

Generally, the field of the invention relates to catalysts containing molybdenum, vanadium, antimony and niobium which have been shown to be effective for conversion of propane to acrylonitrile and isobutane to methacrylonitrile (via an ammoxidation reaction) and/or for conversion of propane to acrylic acid and isobutane to methacrylic acid (via an oxidation reaction). The art known in this field includes numerous patents and patent applications, including for example, U.S. Pat. No. 5,750,760 to Ushikubo et al., U.S. Pat. No. 6,036,880 to Komada et al., U.S. Pat. No. 6,143,916 to Hinago et al., U.S. Pat. No. 6,514,902 to Inoue et al., U.S. Patent Application No. US 2003/0088118 A1 by Komadu et al., U.S. Patent Application No. 2004/0063990 A1 to Gaffney et al., PCT Patent Application No. WO 2004/108278 A1 by Asahi Kasei Kabushiki Kaisha, Japanese Patent Application No. JP 1999/114426 A by Asahi Chemical Co., and Japanese Patent Application No. JP 2000/1126599 A by Asahi Chemical Co.

Although advancements have been made in the art in connection with catalysts containing molybdenum, vanadium, antimony and niobium effective for conversion of propane to acrylonitrile and isobutane to methacrylonitrile (via an ammoxidation reaction) and/or for conversion of propane to acrylic acid and isobutane to methacrylic acid (via an oxidation reaction), the catalysts need further improvement before becoming commercially viable. In general, the art-known catalytic systems for such reactions suffer from generally low yields of the desired product. Also, the synthesis procedures known in the art for such catalyst systems are difficult to reproduce in a manner that leads to consistency in catalyst performance.

### SUMMARY OF THE INVENTION

The present invention relates to catalyst compositions comprising molybdenum, vanadium, antimony, niobium, at least one element selected from the group consisting of titanium, tin, zirconium, and hafnium, and at least one lanthanide selected from the group consisting of lanthanum, praseodymium, neodymium, samarium, europium, gadolinium, terbium, dysprosium, holmium, erbium, thulium, ytterbium, and lutetium

In one embodiment, the invention is a catalyst composition comprising a mixed oxide of empirical formula:

MoVₐSb_{b}Nb_{c}X_{d}LₑOₙ

wherein X is selected from the group consisting of Ti, Sn, Zr, Hf, and mixtures thereof,
L is selected from the group consisting of La, Pr, Nd, Sm, Eu, Gd, Th, Dy,
Ho, Er, Tm, Yb, Lu and mixtures thereof,
0.1<a<0.8,
0.01<b<0.6,
0.01<c<0.2,
0.005<d<0.6,
0<e<0.04; and
n is number of oxygen atoms required to satisfy valance requirements of all other elements present in the mixed oxide with the proviso that one or more of the other elements in the mixed oxide can be present in an oxidation state lower than its highest oxidation state, and
a, b, c, d, and e represent the molar ratio of the corresponding element to one mole of Mo.

In other embodiments X is Ti, Sn or mixtures thereof.

In other embodiments, L is Nd or Pr.

The present invention also relates to a process for the ammoxidation of a saturated or unsaturated or mixture of saturated and unsaturated hydrocarbon to produce an unsaturated nitrile, the process comprising contacting the saturated or unsaturated or mixture of saturated and unsaturated hydrocarbon with ammonia and an oxygen-containing gas in the presence the catalyst compositions described herein. In one embodiment, the present invention is a process for the conversion of a hydrocarbon selected from the group consisting of propane, isobutane or mixtures thereof, to acrylonitrile, methacrylonitrile, or mixtures thereof, the process comprising the step of reacting in the vapor phase at an elevated temperature and pressure said hydrocarbon with a molecular oxygen-containing gas and ammonia, in the presence of the catalyst compositions described herein.

### DETAILED DESCRIPTION OF THE INVENTION

The present invention generally relates to catalyst compositions, methods of preparing such catalyst compositions, and methods of using such catalyst compositions. Such compositions and such catalysts are effective for the ammoxidation of propane to acrylonitrile and isobutane to methacrylonitrile and/or for the oxidation of propane to acrylic acid and isobutane to methacrylic acid.

### Catalyst Composition

In one embodiment, the invention is a catalyst composition comprising molybdenum, vanadium, antimony, niobium, at least one element selected from the group consisting of titanium, tin, germanium, zirconium, and hafnium, and at least one lanthanide selected from the group consisting of lanthanum, praseodymium, neodymium, samarium, europium, gadolinium, dysprosium, holmium, erbium, thulium, ytterbium, and lutetium. As used herein, “at least one element selected from the group . . . ” or “at least one lanthanide selected from the group . . . ” includes within in its scope mixtures of two or more of the listed elements or lanthanides, respectively.

In one embodiment, the invention is a catalyst composition comprising a mixed oxide of empirical formula:

MoVₐSb_{b}Nb_{c}X_{d}LₑOₙ

wherein X is selected from the group consisting of Ti, Sn, Zr, Hf, and mixtures thereof;
L is selected from the group consisting of La, Pr, Nd, Sm, Eu, Gd, Th, Dy, Ho, Er, Tm, Yb, Lu and mixtures thereof;
0.1<a<0.8,
0.01<b<0.6,
0.01<c<0.2,
0.005<d<0.6,
0<e<0.04; and
n is number of oxygen atoms required to satisfy valance requirements of all other elements present in the mixed oxide with the proviso that one or more of the other elements in the mixed oxide can be present in an oxidation state lower than its highest oxidation state, and,
a, b, c, d and e represent the molar ratio of the corresponding element to one mole of Mo.

In one embodiment, the invention is a catalyst composition comprising a mixed oxide of empirical formula:

MoVₐSb_{b}Nb_{c}X_{d}LₑOₙ

wherein X is selected from the group consisting of Ti, Sn, and mixtures thereof;
L is selected from the group consisting of La, Pr, Nd, Sm, Eu, Gd, Tb, Dy, Ho, Er, Tm, Yb, Lu and mixtures thereof;
0.1<a<0.8,
0.01<b<0.6,
0.02<c<0.2,
0.005<d<0.6,
0<e<0.04; and
n is number of oxygen atoms required to satisfy valance requirements of all other elements present in the mixed oxide with the proviso that one or more of the other elements in the mixed oxide can be present in an oxidation state lower than its highest oxidation state, and
a, b, c, d and e represent the molar ratio of the corresponding element to one mole of Mo.

In other embodiments of the catalyst compositions described by the above empirical formulas X is one of Ti or Sn. In other embodiments of the catalyst compositions described by the above empirical formulas, X is Ti, X is Sn, X is Zr, and X is Hf.

In other embodiments of the catalyst compositions described by the above empirical formulas, L is one of Nd or Pr. In other embodiments of the catalyst compositions described by the above empirical formulas, L is La, L is Pr, L is Nd, L is Sm, L is Eu, L is Gd, L is Th, L is Dy, L is Ho, L is Er, L is Tm, L is Yb, and L is Lu.

In other embodiment of the catalyst compositions described by the above empirical formulas, the catalyst composition contains no cerium. In another embodiment of the catalyst compositions described by the above empirical formulas, the catalyst composition contains no tellurium. In another embodiment of the catalyst compositions described by the above empirical formulas, the catalyst composition contains no tantalum. In another embodiment of the catalyst compositions described by the above empirical formulas, the catalyst composition contains no germanium.

In other embodiments of the catalyst compositions described by the above empirical formulas, a, b, c, d, and e are each independently within the following ranges: 0.1<a, 0.2<a, a<0.4, a<0.8, a<0.3, 0.1<b, b<0.3, b<0.6, 0.02<c, 0.03<c, 0.04<c, c<0.2, c<0.15, c<0.12, c<0.1, 0.005<d, 0.01<d, d<0.6, d<0.3, d<0.2, d<0.15, d<0.1, d<0.05 and 0<e, 0.001<e, e<0.04, e<0.02, e<0.016, e<0.01, e<0.006.

In one embodiment of the catalyst compositions described by the above empirical formulas, the catalyst additionally contains lithium and may optionally contain one or more other alkali metals. In this embodiment the catalyst composition comprises a mixed oxide of the empirical formula

MoVₐSb_{b}Nb_{c}X_{d}LₑA_{f}Li_{g}Oₙ

Wherein X, L, a, b, c, d, e, and n are previously described herein, A is at least one of Na, K, Cs, Rb and mixtures thereof, 0≦f<0.1, 0<g<0.1, and “f” and “g” represent the molar ratio of the corresponding element to one mole of Mo. In another embodiments of the catalyst composition comprising a mixed oxide described by the above empirical formula, the catalyst composition contains no Na, K, Cs, Rb or mixtures thereof (i.e. f equals 0). In other embodiments of the catalyst composition comprising a mixed oxide described by the above empirical formula, 0<g, 0.03<g, g<0.06, g<0.1.

The catalyst of the present invention may be made and used either supported or unsupported (i.e. the catalyst may comprise a support or may be a bulk catalyst). Suitable supports are silica, alumina, zirconia, titania, or mixtures thereof. However, when zirconia or titania are used as support materials then the ratio of molybdenum to zirconium or titanium increases over the values shown in the above formulas, such that the Mo to Zr or Ti ratio is between about 1:1 to 1:10. A support typically serves as a binder for the catalyst resulting in a harder and more attrition resistant catalyst. However, for commercial applications, an appropriate blend of both the active phase (i.e. the complex of catalytic oxides described above) and the support is helpful to obtain an acceptable activity and hardness (attrition resistance) for the catalyst. Directionally, any increase in the amount of the active phase decreases the hardness of the catalyst. The support comprises between 10 and 90 weight percent of the supported catalyst. Typically, the support comprises between 40 and 60 weight percent of the supported catalyst. In one embodiment of this invention, the support may comprise as little as about 10 weight percent of the supported catalyst. In one embodiment of this invention, the support may comprise as little as about 30 weight percent of the supported catalyst. In another embodiment of this invention, the support may comprise as much as about 70 weight percent of the supported catalyst. Support materials are available which may contain one or more promoter elements, e.g. a silica sol containing sodium (Na), and such promoter elements may be incorporated into the catalyst via the support material.

In one embodiment the catalyst is supported using a silica sol. If the average colloidal particle diameter of said silica sol is too small, the surface area of the manufactured catalyst will be increased and the catalyst will exhibit reduced selectivity. If the colloidal particle diameter is too large, the manufactured catalyst will have poor anti-abrasion strength. Typically, the average colloidal particle diameter of the silica sol is between about 15 nm and about 50 nm. In one embodiment of this invention, the average colloidal particle diameter of the silica sol is about 10 nm and can be as low as about 8 nm. In another embodiment of this invention, the average colloidal particle diameter of the silica sol is about 100 nm. In another embodiment of this invention, the average colloidal particle diameter of the silica sol is about 20 nm.

### Catalyst Preparation

The catalyst compositions described herein can be prepared by the hydrothermal synthesis methods described herein. Hydrothermal synthesis methods are disclosed in U.S. Patent Application No. 2003/0004379 to Gaffney et al., Watanabe et al., “New Synthesis Route for Mo—V—Nb—Te mixed oxides catalyst for propane ammoxidation”, Applied Catalysis A: General, 194-195, pp. 479-485 (2000), and Ueda et al., “Selective Oxidation of Light Alkanes over hydrothermally synthesized Mo—V—M-O (M=Al, Ga, Bi, Sb and Te) oxide catalysts.”, Applied Catalysis A: General, 200, pp. 135-145, which are incorporated here by reference.

In general, the catalyst compositions described herein can be prepared by hydrothermal synthesis where source compounds (i.e. compounds which contain and/or provide one or more of the metals for the mixed metal oxide catalyst composition) are admixed in an aqueous solution to form a reaction medium and reacting the reaction medium at elevated pressure and elevated temperature in a sealed reaction vessel for a time sufficient to form the mixed metal oxide. In one embodiment, the hydrothermal synthesis continues for a time sufficient to fully react any organic compounds present in the reaction medium, for example, solvents used in the preparation of the catalyst or any organic compounds added with any of the source compounds supplying the mixed metal oxide components of the catalyst composition. This embodiment simplifies further handling and processing of the mixed metal oxide catalyst.

The source compounds are reacted in the sealed reaction vessel at a temperature greater than 100° C. and at a pressure greater than ambient pressure to form a mixed metal oxide precursor. In one embodiment, the source compounds are reacted in the sealed reaction vessel at a temperature of at least about 125° C., in another embodiment at a temperature of at least about 150° C., and in yet another embodiment at a temperature of at least about 175° C. In one embodiment, the source compounds are reacted in the sealed reaction vessel at a pressure of at least about 25 psig, and in another embodiment at a pressure of at least about 50 psig, and in yet another embodiment at a pressure of at least about 100 psig. Such sealed reaction vessels may be equipped with a pressure control device to avoid over pressurizing the vessel and/or to regulate the reaction pressure.

In any case, the source compounds are preferably reacted by a protocol that comprises mixing the source compounds during the reaction step. The particular mixing mechanism is not critical, and can include for example, mixing (e.g., stirring or agitating) the components during the reaction by any effective method. Such methods include, for example, agitating the contents of the reaction vessel, for example by shaking, tumbling or oscillating the component-containing reaction vessel. Such methods also include, for example, stirring by using a stirring member located at least partially within the reaction vessel and a driving force coupled to the stirring member or to the reaction vessel to provide relative motion between the stirring member and the reaction vessel. The stirring member can be a shaft-driven and/or shaft-supported stirring member. The driving force can be directly coupled to the stirring member or can be indirectly coupled to the stirring member (e.g., via magnetic coupling). The mixing is generally preferably sufficient to mix the components to allow for efficient reaction between components of the reaction medium to form a more homogeneous reaction medium (e.g., and resulting in a more homogeneous mixed metal oxide precursor) as compared to an unmixed reaction. This results in more efficient consumption of starting materials and in a more uniform mixed metal oxide product. Mixing the reaction medium during the reaction step also causes the mixed metal oxide product to form in solution rather than on the sides of the reaction vessel. This allows more ready recovery and separation of the mixed metal oxide product by techniques such as centrifugation, decantation, or filtration and avoids the need to recover the majority of product from the sides of the reactor vessel. More advantageously, having the mixed metal oxide form in solution allows for particle growth on all faces of the particle rather than the limited exposed faces when the growth occurs out from the reactor wall.

It is generally desirable to maintain some headspace in the reactor vessel. The amount of headspace may depend on the vessel design or the type of agitation used if the reaction mixture is stirred. Overhead stirred reaction vessels, for example, may take 50% headspace. Typically, the headspace is filled with ambient air which provides some amount of oxygen to the reaction. However, the headspace, as is known the art, may be filled with other gases to provide reactants like O₂ or even an inert atmosphere such as Ar or N₂. The amount of headspace and gas within it depends upon the desired reaction as is known in the art.

The source compounds can be reacted in the sealed reaction vessel at an initial pH of not more than about 4. Over the course of the hydrothermal synthesis, the pH of the reaction mixture may change such that the final pH of the reaction mixture may be higher or lower than the initial pH. Preferably, the source compounds are reacted in the sealed reaction vessel at a pH of not more than about 3.5. In some embodiments, the components can be reacted in the sealed reaction vessel at a pH of not more than about 3.0, of not more than about 2.5, of not more than about 2.0, of not more than about 1.5 or of not more than about 1.0, of not more than about 0.5 or of not more than about 0. Preferred pH ranges include a pH ranging from about negative 0.5 to about 4, preferably from about 0 to about 4, more preferably from about 0.5 to about 3.5. In some embodiments, the pH can range from about 0.7 to about 3.3, or from about 1 to about 3. The pH may be adjusted by adding acid or base to the reaction mixture.

The source compounds can be reacted in the sealed reaction vessels at the aforementioned reaction conditions (including for example, reaction temperatures, reaction pressures, pH, stirring, etc., as described above) for a period of time sufficient to form the mixed metal oxide, preferably where the mixed metal oxide comprises a solid state solution comprising the required elements as discussed above, and at least a portion thereof preferably having the requisite crystalline structure for active and selective propane or isobutane oxidation and/or ammoxidation catalysts, as described below. The exact period of time is not narrowly critical, and can include for example at least about six hours, at least about twelve hours, at least about eighteen hours, at least about twenty-four hours, at least about thirty hours, at least about thirty-six hours, at least about forty-two hours, at least about forty-eight hours, at least about fifty-four hours, at least about sixty hours, at least about sixty-six hours or at least about seventy-two hours. Reaction periods of time can be even more than three days, including for example at least about four days, at least about five days, at least about six days, at least about seven days, at least about two weeks or at least about three weeks or at least about one month.

Following the reaction step, further steps of the preferred catalyst preparation methods can include work-up steps, including for example cooling the reaction medium comprising the mixed metal oxide (e.g., to about ambient temperature), separating the solid particulates comprising the mixed metal oxide from the liquid (e.g., by centrifuging and/or decanting the supernatant, or alternatively, by filtering), washing the separated solid particulates (e.g., using distilled water or deionized water), repeating the separating step and washing steps one or more times, and effecting a final separating step. In one embodiment, the work up step comprises drying the reaction medium, such as by rotary evaporation, spray drying, freeze drying etc. This eliminates the formation of a metal containing waste stream.

After the work-up steps, the washed and separated mixed metal oxide can be dried. Drying the mixed metal oxide can be effected under ambient conditions (e.g., at a temperature of about 25° C. at atmospheric pressure), and/or in an oven, for example, at a temperature ranging from about 40° C. to about 150° C., and preferably of about 120° C. over a drying period of about time ranging from about five to about fifteen hours, and preferably of about twelve hours. Drying can be effected under a controlled or uncontrolled atmosphere, and the drying atmosphere can be an inert gas, an oxidative gas, a reducing gas or air, and is typically and preferably air.

As a further preparation step, the dried mixed metal oxide can be treated to form the mixed metal oxide catalyst. Such treatments can include for example calcinations (e.g., including heat treatments under oxidizing or reducing conditions) effected under various treatment atmospheres. The work-up mixed metal oxide can be crushed or ground prior to such treatment, and/or intermittently during such pretreatment. Preferably, for example, the dried mixed metal oxide can be optionally crushed, and then calcined to form the mixed metal oxide catalyst. The calcination is preferably effected in an inert atmosphere such as nitrogen. Preferred calcination conditions include temperatures ranging from about 400° C. to about 700° C., more preferably from about 500° C. to about 650° C., and in some embodiments, the calcination can be at about 600° C.

The treated (e.g., calcined) mixed metal oxide can be further mechanically treated, including for example by grinding, sieving and pressing the mixed metal oxide into its final form for use in fixed bed or fluid bed reactors. In other embodiments, the catalyst may be shaped into its final form prior to any calcinations or other heat treatment. For example, in the preparation of a fixed bed catalyst, the catalyst precursor slurry is typically dried by heating at an elevated temperature and then shaped (e.g. extruded, pellitized, etc.) to the desired fixed bed catalyst size and configuration prior to calcination. Similarly, in the preparation of fluid bed catalysts, the catalyst precursor slurry is may be spray dried to yield microspheroidal catalyst particles having particle diameters in the range from 10 and 200 microns and then be calcined.

Some source compounds containing and providing the metal components used in the synthesis of the catalyst (also referred to herein as a “source” or “sources”) can be provided to the reaction vessel as aqueous solutions of the metal salts. Some source compounds of the metal components can be provided to the reaction vessels as solids or as slurries comprising solid particulates dispersed in an aqueous media. Some source compounds of the metal components can be provided to the reaction vessels as solids or as slurries comprising solid particulates dispersed in an non-aqueous solvents or other non-aqueous media.

Suitable source compounds for synthesis of the catalysts as described herein include the following. A suitable molybdenum source may include molybdenum(VI) oxide (MoO₃), ammonium heptamolybdate or molybdic acid. A suitable vanadium source may include vanadyl sulfate, ammonium metavanadate or vanadium(V) oxide. A suitable antimony source may include antimony(III) oxide, antimony(III) acetate, antimony(III) oxalate, antimony(V) oxide, antimony(III) sulfate, or antimony(III) tartrate. A suitable niobium source may include niobium oxalate, ammonium niobium oxalate, niobium oxide, niobium ethoxide or niobic acid.

A Suitable titanium source may include rutile and/or anatase titanium dioxide (TiO₂), e.g. Degussa P-25, titanium isopropoxide, TiO(oxalate), TiO(acetylacetonate)₂, or titanium alkoxide complexes, such as Tyzor 131. A suitable tin source may include tin (II) acetate. A suitable germanium source may include germanium(IV) oxide. A suitable zirconium source may include zirconyl nitrate or zirconium (IV) oxide. A suitable hafnium sources may include hafnium (IV) chloride or hafnium (IV) oxide.

Suitable lanthanum sources may include lanthanum (III) chloride or lanthanum (III) oxide, and preferred is lanthanum (III) acetate hydrate. Suitable praseodymium sources may include praseodymium (III) chloride, praseodymium (III, IV) oxide or praseodymium (III) isopropoxide, and preferred is praseodymium (III) acetate hydrate. Suitable neodymium sources may include neodymium (III) chloride, neodymium (III) oxide or neodymium (III) isopropoxide, and preferred is neodymium (III) acetate hydrate. Suitable samarium sources may include samarium (III) chloride, samarium (II) oxide or samarium (III) isopropoxide, and preferred is samarium (III) acetate hydrate. Suitable europium sources may include europium (III) chloride or europium (III) oxide, and preferred is europium (III) acetate hydrate. Suitable gadolinium sources may include gadolinium (III) chloride or gadolinium (III) oxide, and preferred is gadolinium (III) acetate hydrate. Suitable terbium sources include terbium (III) chloride or terbium (III) oxide, and preferred is terbium (III) acetate hydrate. Suitable dysprosium sources may include dysprosium (III) chloride, dysprosium (III) oxide or dysprosium (III) isopropoxide, and preferred is dysprosium (III) acetate hydrate. Suitable holmium sources may include holmium (III) chloride, holmium (III) oxide or holmium (III) acetate hydrate (preferred). Suitable erbium sources may include erbium (III) chloride, erbium (III) oxide or erbium (III) isopropoxide, and preferred is erbium (III) acetate hydrate (preferred). Suitable thulium sources may include thulium (III) chloride or thulium (III) oxide, and preferred is thulium (III) acetate hydrate. Suitable ytterbium sources may include ytterbium (III) chloride, ytterbium (III) oxide or ytterbium (III) isopropoxide, and preferred is ytterbium (III) acetate hydrate. Suitable sources of lutetium may include lutetium (III) chloride or lutetium (III) oxide, and preferred is lutetium (III) acetate hydrate. Nitrates of the above listed metals may also be employed as source compounds.

Solvents which may be used to prepare mixed metal oxides according to the invention include, but are not limited to, water, alcohols such as methanol, ethanol, propanol, diols (e.g. ethylene glycol, propylene glycol, etc.), organic acids such as acetic acid, as well as other polar solvents known in the art. The metal source compounds are at least partially soluble in the solvent, at least at the reaction temperature and pressure, and preferably, the metal source compounds are slightly soluble in the solvent. Generally, water is the preferred solvent. Any water suitable for use in chemical synthesis may be used. The water may, but need not be, distilled and/or deionized.

The amount of aqueous solvent in the reaction medium may vary due to the solubilities of the source compounds combined to form the particular mixed metal oxide. The amount of aqueous solvent should at least be sufficient to yield a slurry ((a mixture of solids and liquids which is able to be stirred) of the reactants. It is typical in hydrothermal synthesis of mixed metal oxides to leave an amount of headspace in the reactor vessel.

In some hydrothermal synthesis methods an oxidant may be added to the reaction medium to oxidize one or more of the metal precursors prior to the reaction step. For example, in the hydrothermal preparation of the catalyst compositions described herein, some of the vanadium and antimony may be oxidized with an oxidant prior to the reaction step. In that case oxidant, such as H₂O₂, is added to the reaction medium. This is preferably done prior to addition of the niobium precursor compound, e.g. niobium oxalate, to avoid unwanted reaction of the H₂O₂ with oxalic acid with the niobium oxalate solution. Thus, when an oxidant is added to the reaction medium the order of addition may be chosen to achieve the desired oxidation and/or to avoid undesired reactions. The oxidant is preferably a non-metal-containing oxide such as H₂O₂. Metal-containing or inorganic oxidants may be used when it is desirable to introduce the particular metals or elements of the oxidant into the mixed metal oxide.

Variations on the above methods will be recognized by those skilled in the art. For example a method for preparing the catalyst described herein having the following empirical formula:

MoV_{0.1-0.3}Sb_{0.1-0.3}Nb_{0.03-0.15}Ti_{0.05-0.25}LₑOₙ

in which L is La, Pr, Nd, Sm, Eu, Gd, Th, Dy, Ho, Er, Tm, Yb, Lu and mixtures thereof, “e” is greater than zero and less than about 0.02, and “n” is determined by the oxidized states of the other elements, comprises preparing solutions or slurries of source compounds for the catalyst. In one or the first slurry, molybdenum trioxide (MoO₃), vanadium oxysulfate (VOSO₄), antimony oxide (Sb₂O₃), titanium dioxide (TiO₂), at least one “L” source compound are dissolved/slurried in water at the desired ratios (all ratios are relative to molybdenum metal). In the other or second solution or slurry, niobic acid (Nb₂O₅.nH₂O) is mixed with oxalic acid (HO₂CCO₂H). A range of oxalic acid:niobium molar ratios may be employed. In one embodiment the oxalic acid:niobium molar ratio is about 3:1. The two solutions/slurries are combined with one another, heated with mixing to 175° C. and held at this temperature for 48 hours, and then cooled to room temperature, typically by natural heat dissipation. The cooled slurry is filtered to remove the mother liquor, and the remaining solids are washed and then dried and then calcined under nitrogen at 600° C. to activate the catalyst. The calcined catalyst is pulverized, then pelletized and sized, or spray dried for testing and/or ultimate use.

Another and preferred technique useful for the preparation of catalysts described herein, is the use of vanadium oxide (V₂O₅) as the vanadium source in the preparation of such catalyst compositions. This substitution results in, among other things, a solution/slurry that after mixing with the oxalic acid/niobium solution/slurry does not require filtering and/or washing (as opposed to using other vanadium sources such as vanadium oxysulfate). Rather, the resulting solution/slurry can simply be dried and calcined. Another benefit of using V₂O₅ (compared to the use of VOSO₄ as described above) is lower corrosion of the hydrothermal synthesis reactor components. Although the pH of the sulfate and sulfate free slurries are similar, the corrosion rate of exposed stainless steel differs substantially for the two syntheses.

In one embodiment of this particular preparation method (i.e. using vanadium oxide (V₂O₅) as the vanadium source and niobic acid as the niobium source), the oxalic acid to niobium molar ratio of the solution/slurry is at least 4.5:1, preferably at least 4.7:1. Using oxalate as a solubilizing agent allows more precise control of the degree of reduction of the catalyst as it is being formed and dramatically increases the yield of solid product from the hydrothermal synthesis.

Another advantage of using oxalate is that it decomposes during the hydrothermal synthesis so that there are no species that need to be washed from the catalyst after the hydrothermal synthesis. By adjusting the nominal composition of the hydrothermal synthesis to match the target catalyst composition, the wash step can be eliminated while still obtaining a catalyst with high performance. The benefits are that the yield of the hydrothermal synthesis is 100% on a metals basis, filtration steps can be eliminated from the synthesis, and no vanadium or molybdenum containing aqueous waste streams are produced from the synthesis. These factors are particularly important when the catalyst synthesis is scaled to a pilot plant or plant scale.

As such, in one embodiment, the invention is a process for preparing a mixed metal oxide catalyst comprising molybdenum, vanadium, antimony, niobium, at least one element select from the group consisting of titanium, tin, germanium, zirconium, and hafnium and at least one lanthanide selected from the group consisting of lanthanum, cerium, praseodymium, neodymium, samarium, europium, gadolinium, terbium, dysprosium, holmium, erbium, thulium, ytterbium and lutetium, wherein the process comprising mixing and reacting source compounds of the metal components and wherein the vanadium source is vanadium oxide (V₂O₅). In another embodiment, such a process comprises the following steps:
(i) preparing a first mixture comprising source compounds of Mo, V, Sb at least one of Ti, Sn, Ge, Zr, or Hf, and at least one of at least one of La, Ce, Pr, Nd, Sm, Eu, Gd, Th, Dy, Ho, Er, Tm, Yb, Lu, wherein the V source compound is vanadium oxide (V₂O₅);
(ii) preparing a second mixture comprising a niobium source compound and oxalic acid (H₂O₂CCO₂H) wherein the oxalic acid to niobium molar ratio is at least 4.5 to 1;
(iii) admixing, in a reaction vessel said first mixture and said second mixture in an aqueous solvent to form a reaction medium;
(iv) optionally adding additional aqueous solvent to the reaction vessel;
(v) sealing the reaction vessel;
(vi) reacting the reaction medium at a temperature greater than 100° C. and a pressure greater than ambient pressure for a time sufficient to form a mixed metal oxide;
(vii) optionally cooling the reaction medium; and
(viii) drying and calcining the mixed metal oxide

### Conversion of Propane and Isobutane via Ammoxidation and Oxidation Reactions

Propane is preferably converted to acrylonitrile and isobutane to methacrylonitrile, by providing one or more of the aforementioned catalysts in a gas-phase flow reactor, and contacting the catalyst with propane or isobutane in the presence of oxygen (e.g. provided to the reaction zone in a feedstream comprising an oxygen-containing gas, such as and typically air) and ammonia under reaction conditions effective to form acrylonitrile or methacrylonitrile. For this reaction, the feed stream preferably comprises propane or isobutane, an oxygen-containing gas such as air, and ammonia with the following molar ratios of: propane or isobutane to oxygen in a ratio ranging from about 0.125 to about 5, and preferably from about 0.25 to about 2.5, and propane or isobutane to ammonia in a ratio ranging from about 0.3 to about 2.5, and preferably from about 0.5 to about 2.0. The feed stream can also comprise one or more additional feed components, including acrylonitrile or methacrylonitrile product (e.g., from a recycle stream or from an earlier-stage of a multi-stage reactor), and/or steam. For example, the feedstream can comprise about 5% to about 30% by weight relative to the total amount of the feed stream, or by mole relative to the amount of propane or isobutane in the feed stream. In one embodiment the catalyst compositions described herein are employed in the ammoxidation of propane to acrylonitrile is a once-through process, i.e., it operates without recycle of recovered but unreacted feed materials.

Propane can also be converted to acrylic acid and isobutane to methacrylic acid by providing one or more of the aforementioned catalysts in a gas-phase flow reactor, and contacting the catalyst with propane in the presence of oxygen (e.g. provided to the reaction zone in a feedstream comprising an oxygen-containing gas, such as and typically air) under reaction conditions effective to form acrylic acid. The feed stream for this reaction preferably comprises propane and an oxygen-containing gas such as air in a molar ratio of propane or isobutane to oxygen ranging from about 0.15 to about 5, and preferably from about 0.25 to about 2. The feed stream can also comprise one or more additional feed components, including acrylic acid or methacrylic acid product (e.g., from a recycle stream or from an earlier-stage of a multi-stage reactor), and/or steam. For example, the feedsteam can comprise about 5% to about 30% by weight relative to the total amount of the feed stream, or by mole relative to the amount of propane or isobutane in the feed stream.

The specific design of the gas-phase flow reactor is not narrowly critical. Hence, the gas-phase flow reactor can be a fixed-bed reactor, a fluidized-bed reactor, or another type of reactor. The reactor can be a single reactor, or can be one reactor in a multi-stage reactor system. Preferably, the reactor comprises one or more feed inlets for feeding a reactant feedstream to a reaction zone of the reactor, a reaction zone comprising the mixed metal oxide catalyst, and an outlet for discharging reaction products and unreacted reactants.

The reaction conditions are controlled to be effective for converting the propane to acrylonitrile or acrylic acid, or for converting the isobutane to methacrylonitrile or methacrylic acid. Generally, reaction conditions include a temperature ranging from about 300° C. to about 550° C., preferably from about 325° C. to about 500° C., and in some embodiments from about 350° C. to about 450° C., and in other embodiments from about 430° C. to about 520° C. Generally, the flow rate of the propane or isobutene containing feedstream through the reaction zone of the gas-phase flow reactor can be controlled to provide a weight hourly space velocity (WHSV) ranging from about 0.02 to about 5, preferably from about 0.05 to about 1, and in some embodiments from about 0.1 to about 0.5, in each case, for example, in grams propane or isobutane to grams of catalyst. The pressure of the reaction zone can be controlled to range from about 0 psig to about 200 psig, preferably from about 0 psig to about 100 psig, and in some embodiments from about 0 psig to about 50 psig.

The resulting acrylonitrile and/or acrylic acid or methacrylonitrile and/or methacrylic acid product can be isolated, if desired, from other side-products and/or from unreacted reactants according to methods known in the art.

The catalyst compositions described herein when employed in the single pass (i.e. no recycle) ammoxidation of propane are capable of producing a yield of about 57-58 percent acrylonitrile, with a selectivity of about 24% to COX (carbon dioxide+carbon monoxide), and a selectivity of about 13% to a mixture of hydrogen cyanide (HCN) and acetonitrile or methyl cyanide (CH₃CN). The effluent of the reactor may also include unreacted oxygen (O₂), ammonia (NH₃) and entrained catalyst fines

### Specific Embodiments

In order to illustrate the instant invention, samples of a base catalyst, with and without various catalyst modifiers, were prepared and then evaluated under similar reaction conditions. The compositions listed below are nominal compositions, based on the total metals added in the catalyst preparation. Since some metals may be lost or may not completely react during the catalyst preparation, the actual composition of the finished catalyst may vary slightly from the nominal compositions shown below.

### EXAMPLE #1

### Mo₁V_{0.3}Sb_{0.2}Nb_{0.06}Ti_{0.1}Tb_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Tb(OAc)₃ (0.875 mL of a 0.04 M solution), niobium oxalate (2.1 mL of a 0.1 M solution) and water (2.98 mL). As used in this example and several subsequent examples, “(OAc)₃” designates the acetate hydrate for the named lanthanide metal. The reactor was then sealed with a Teflon cap in a metal housing, placed in an oven preheated to 175° C. and continuously rotated to affect mixing of the liquid and solid reagents. After 48 h (h=hours), the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.01}Tb_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 53% was obtained (83% propane conversion, 64% acrylonitrile selectivity). At 420° C., WHSV=0.2 and a feed ratio of C₃H₈/NH₃/O₂/He=1/1.4/3/12, an acrylonitrile yield of 45% was obtained (65% propane conversion, 70% acrylonitrile selectivity).

### EXAMPLE #2

### Mo₁V_{0.3}Sb_{0.2}Nb_{0.06}Ti_{0.1}Tb_{0.01}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Tb(OAc)₃ (1.750 mL of a 0.04 M solution), niobium oxalate (2.1 mL of a 0.1 M solution) and water (2.1 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Tb_{0.01}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 58% was obtained (87% propane conversion, 66% acrylonitrile selectivity). At 420° C., WHSV=0.2 and a feed ratio of C₃H₈/NH₃/O₂/He=1/1.4/3/12, an acrylonitrile yield of 52% was obtained (76% propane conversion, 69% acrylonitrile selectivity).

### EXAMPLE #3

### Mo₁V_{0.3}Sb_{0.2}Nb_{0.06}Ti_{0.1}Nd_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Nd(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Nd_{0.01}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 55% was obtained (87% propane conversion, 63% acrylonitrile selectivity). At 420° C., WHSV=0.2 and a feed ratio of C₃H₈/NH₃/O₂/He=1/1.4/3/12, an acrylonitrile yield of 53% was obtained (80% propane conversion, 67% acrylonitrile selectivity).

### EXAMPLE #4

### Mo₁V_{0.3}Sb_{0.2}Nb_{0.06}Ti_{0.1}Nd_{0.01}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Nd(OAc)₃ (1.750 mL of a 0.04 M solution), niobium oxalate (2.1 mL of a 0.1 M solution) and water (2.1 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Nd_{0.01}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 36% was obtained (79% propane conversion, 45% acrylonitrile selectivity). At 420° C., WHSV=0.2 and a feed ratio of C₃H₈/NH₃/O₂/He=1/1.4/3/12, an acrylonitrile yield of 40% was obtained (68% propane conversion, 58% acrylonitrile selectivity).

### EXAMPLE #5

### Mo₁V_{0.3}Sb_{0.2}Nb_{0.06}Ti_{0.1}Nd_{0.05}Tb_{0.005}Oₙ

A 23 mL Teflon reactor liner was loaded with MoO₃ (1.0 g), VOSO₄ (2.002 mL of a 1.041 M solution)), Sb₂O₃ (2.824 mL of a 0.246 M slurry), TiO₂ (2.48 mL of a 0.280 M slurry), Nd(OAc)₃ (0.877 mL of a 0.04 M solution), Tb(OAc)₃ (0.853 mL of a 0.04 M solution niobium oxalate (2.084 mL of a 0.1 M solution) and water (2.1 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Nd_{0.005}Tb_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 53% was obtained (94% propane conversion, 56% acrylonitrile selectivity). At 420° C., WHSV=0.2 and a feed ratio of C₃H₈/NH₃/O₂/He=1/1.4/3/12, an acrylonitrile yield of 53% was obtained (88% propane conversion, 60% acrylonitrile selectivity).

### EXAMPLE #6

### Mo₁V_{0.3}Sb_{0.2}Nb_{0.06}Ti_{0.1}Sm_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Sm(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Sm_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 47% was obtained (88% propane conversion, 54% acrylonitrile selectivity). At 420° C., WHSV=0.2 and a feed ratio of C₃H₈/NH₃/O₂/He=1/1.4/3/12, an acrylonitrile yield of 48% was obtained (86% propane conversion, 56% acrylonitrile selectivity).

### EXAMPLE #7

### Mo₁V_{0.3}Sb_{0.2}Nb_{0.06}Ti_{0.1}La_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), La(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}La_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 47% was obtained (87% propane conversion, 54% acrylonitrile selectivity). At 420° C., WHSV=0.2 and a feed ratio of C₃H₈/NH₃/O₂/He=1/1.4/3/12, an acrylonitrile yield of 43% was obtained (72% propane conversion, 60% acrylonitrile selectivity).

### EXAMPLE #8

### Mo₁V_{0.3}Sb_{0.2}Nb_{0.06}Ti_{0.1}Pr_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Pr(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Pr_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 51% was obtained (86% propane conversion, 59% acrylonitrile selectivity). At 420° C., WHSV=0.2 and a feed ratio of C₃H₈/NH₃/O₂/He=1/1.4/3/12, an acrylonitrile yield of 44% was obtained (70% propane conversion, 63% acrylonitrile selectivity).

### EXAMPLE #9

### Mo₁V_{0.3}Sb_{0.2}Nb_{0.06}Ti_{0.1}Eu_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Eu(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Eu_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 49% was obtained (90% propane conversion, 54% acrylonitrile selectivity). At 420° C., WHSV=0.2 and a feed ratio of C₃H₈/NH₃/O₂/He=1/1.4/3/12, an acrylonitrile yield of 46% was obtained (72% propane conversion, 64% acrylonitrile selectivity).

### EXAMPLE #10

### Mo₁Vo_{0.3}Sb_{0.2}Nb_{0.06}Ti_{0.1}Gd_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Gd(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Gd_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 31% was obtained (83% propane conversion, 37% acrylonitrile selectivity). At 420° C., WHSV=0.2 and a feed ratio of C₃H₈/NH₃/O₂/He=1/1.4/3/12, an acrylonitrile yield of 27% was obtained (72% propane conversion, 37% acrylonitrile selectivity).

### EXAMPLE #11

### Mo₁V_{0.3}Sb_{0.2}Nb_{0.06}Ti_{0.1}Dy_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Dy(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Dy_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 49% was obtained (89% propane conversion, 55% acrylonitrile selectivity). At 420° C., WHSV=0.2 and a feed ratio of C₃H₈/NH₃/O₂/He=1/1.4/3/12, an acrylonitrile yield of 47% was obtained (77% propane conversion, 62% acrylonitrile selectivity).

### EXAMPLE #12

### Mo₁V_{0.3}Sb_{0.2}Nb_{0.06}Ti_{0.1}H_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Ho(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Ho_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 43% was obtained (90% propane conversion, 48% acrylonitrile selectivity). At 420° C., WHSV=0.2 and a feed ratio of C₃H₈/NH₃/O₂/He=1/1.4/3/12, an acrylonitrile yield of 42% was obtained (70% propane conversion, 59% acrylonitrile selectivity).

### EXAMPLE #13

### Mo₁V_{0.3}Sb_{0.2}Nb_{0.6}Ti_{0.1}Er_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Er(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Er_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 48% was obtained (94% propane conversion, 52% acrylonitrile selectivity). At 420° C., WHSV=0.2 and a feed ratio of C₃H₈/NH₃/O₂/He=1/1.4/3/12, an acrylonitrile yield of 49% was obtained (81% propane conversion, 61% acrylonitrile selectivity).

### EXAMPLE #14

### Mo₁V_{0.3}Sb_{0.2}Nb_{0.06}Ti_{0.1}Tm_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Tm(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Tm_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 50% was obtained (90% propane conversion, 55% acrylonitrile selectivity). At 420° C., WHSV=0.2 and a feed ratio of C₃H₈/NH₃/O₂/He=1/1.4/3/12, an acrylonitrile yield of 46% was obtained (79% propane conversion, 59% acrylonitrile selectivity).

### EXAMPLE #15

### Mo₁V_{0.3}Sb_{0.2}Nb_{0.06}Ti_{0.1}Yb_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Yb(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Yb_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 45% was obtained (87% propane conversion, 51% acrylonitrile selectivity). At 420° C., WHSV=0.2 and a feed ratio of C₃H₈/NH₃/O₂/He=1/1.4/3/12, an acrylonitrile yield of 41% was obtained (84% propane conversion, 49% acrylonitrile selectivity).

### EXAMPLE #16

### Mo₁V_{0.3}Sb_{0.2}Nb_{0.06}Ti_{0.1}Lu_{0.005}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Lu(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Lu_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 54% was obtained (86% propane conversion, 63% acrylonitrile selectivity). At 420° C., WHSV=0.2 and a feed ratio of C₃H₈/NH₃/O₂/He=1/1.4/3/12, an acrylonitrile yield of 51% was obtained (75% propane conversion, 69% acrylonitrile selectivity).

### EXAMPLE #17

### Mo₁V_{0.3}Sb_{0.2}Nb_{0.06}Ti_{0.1}Ce_{0.005}Tb_{0.005}Oₙ

A 23 mL Teflon reactor liner was loaded with MoO₃ (1.0 g), VOSO₄ (2.002 mL of a 1.041 M solution)), Sb₂O₃ (2.824 mL of a 0.246 M slurry), TiO₂ (2.48 mL of a 0.280 M slurry), Ce(OAc)₃ (0.866 mL of a 0.04 M solution), Tb(OAc)₃ (0.853 mL of a 0.04 M solution niobium oxalate (2.084 mL of a 0.1 M solution) and water (2.1 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Ce_{0.005}Tb_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 47% was obtained (95% propane conversion, 49% acrylonitrile selectivity). At 420° C., WHSV=0.2 and a feed ratio of C₃H₈/NH₃/O₂/He=1/1.4/3/12, an acrylonitrile yield of 51% was obtained (86% propane conversion, 60% acrylonitrile selectivity).

### EXAMPLE #18

### Mo₁V_{0.2}Sb_{0.26}Nb_{0.09}Ti_{0.1}Nd_{0.005}Oₙ

To a 23 mL Teflon acid digestion bomb was added MoO₃ (1.20 g), Sb₂O₃ (0.316 g), and V₂O₅ (0.152 g). To the solid mixture was added 2 mL water. The suspension was magnetically stirred for 5 min. To the stirred mixture was added TiO₂ (0.833 mL of an aqueous slurry containing 0.08 g TiO₂ per mL) and Nd(OAc)₃ (0.417 mL of a 0.1M soln.). After another 5 min, a solution of ammonium niobium oxalate (1.50 mL, Nb=0.5M) was added and enough distilled water was added to obtain an 80% fill volume in the reactor. The acid digestion bomb was sealed and placed in an oven preheated to 175° C. and was continuously rotated to affect mixing of the liquid and solid reagents. After 48 h the reactor was cooled. The Teflon liner was removed from the housing and the slurry was transferred to a round bottom flask. The liquid was removed using a rotary evaporator. The solid was further dried in air at 120° C. for 12 h. The solid material was then transferred to a vial, crushed, and calcined for 2 h at 600° C. under N₂. The solid was then ground, pressed, and sieved to a particle size range of 145 to 355 μm and tested for catalytic performance. This material had the nominal composition Mo₁V_{0.2}Sb_{0.26}Nb_{0.09}Ti_{0.1}Nd_{0.005}Oₙ. A 0.15 g portion of the catalyst was diluted with SiC and loaded in a 4 mm ID reactor tube. Under propane ammoxidation conditions, 430° C., WHSV=0.2, and feed ratio C₃H₈/NH₃/O₂/He=1/1.2/3/12 an acrylonitrile yield of 54% was achieved at a propane conversion of 89% and a acrylonitrile selectivity of 61%.

### EXAMPLE #19

### Mo₁V_{0.3}Sb_{0.176}Nb_{0.07}Ti_{0.1}Nd_{0.0051}Li_{0.05}Oₙ

To a 23 mL Teflon lined reactor was added MoO₃ (1.0 g), H₂O (1.46 g), VOSO₄ (0.452 g), Sb₂O₃ (0.178 g), TiO₂ (0.056 g), Nd(OAc)₃ (0.881 g of a 0.4M solution), niobium oxalate (2.429 g of a 0.1 M solution) and Li(OAc), (3.544 g of a 0.01 wt % solution. The reactor was then sealed with a Teflon cap in a metal housing, placed in an oven preheated to 175° C. and continuously rotated to affect mixing of the liquid and solid reagents. After 48 h the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material had the nominal composition Mo₁V_{0.3}Sb_{0.176}Nb_{0.07}Ti_{0.1}Nd_{0.005}Li_{0.05}Oₙ.

The material was tested as a catalyst for the heterogeneous ammoxidation of propane to acrylonitrile. At 440° C., WHSV=0.1 and a feed ratio of C₃H₈/NH₃/O₂/He=1/1.6/3/12, an acrylonitrile yield of 55% was obtained.

### COMPARATIVE EXAMPLE #20

### Mo₁V_{0.3}Sb_{0.2}Nb_{0.06}Ti_{0.1}Ce_{0.0.5}Oₙ

A 23 mL Teflon reactor liner was loaded with MoO₃ (1.0 g), VOSO₄ (2.016 mL of a 1.041 M solution)), Sb₂O₃ (2.849 mL of a 0.246 M slurry), TiO₂ (2.500 mL of a 0.280 M slurry), Ce(OAc)₃ (0.875 mL of a 0.04 M solution), niobium oxalate (2.1 mL of a 0.1 M solution) and water (2.98 mL). The reactor was then sealed with a Teflon cap in a metal housing, placed in an oven preheated to 175° C. and continuously rotated to affect mixing of the liquid and solid reagents. After 48 h, the reactor was cooled and the Teflon liner was removed from the housing. The product slurry was vacuum-filtered using a glass frit and washed three times with 50 mL portions of room temperature water. The wet solid was then dried in air at 90° C. for 12 h. The resulting solid material was crushed and calcined under N₂ for 2 h at 600° C. The solid was then ground, pressed, and sieved to 40-60 mesh and tested for catalytic performance. This material has the nominal composition Mo₁V_{0.3}Sb_{0.2}Nb_{0.06}Ti_{0.1}Ce_{0.005}Oₙ.

The material was tested as a catalyst for the heterogeneous ammoxidation of propane to acrylonitrile. At 420° C., WHSV=0.1 and a feed ratio of C₃H₈/NH₃/O₂/He=1/1.4/3/12, an acrylonitrile yield of 50% was obtained (89% propane conversion, 56% acrylonitrile selectivity). At 420° C., WHSV=0.2 and a feed ratio of C₃H₈/NH₃/O₂/He=1/1.4/3/12, an acrylonitrile yield of 48% was obtained (78% propane conversion, 61% acrylonitrile selectivity).

### COMPARATIVE EXAMPLE #21

### Mo₁V_{0.3}Sb_{0.2}Nb_{0.06}Ge_{0.3}Oₙ

To a 7 mL Teflon acid digestion bomb was added water (1 mL) and MoO₃ (0.5 g). The suspension was magnetically stirred for 5 min. To the stirred mixture was added Sb₂O₃ (0.101 g) and GeO₂ (0.109 g) and after 5 min a solution of VOSO₄ (1.042 mL, 1.0 M). After another 5 min, a solution of Nb oxalate (0.496 mL, 0.42M) was added and enough distilled water was added to obtain an 80% fill volume in the reactor. The acid digestion bomb was sealed and placed in an oven preheated to 175° C. and was continuously rotated to affect mixing of the liquid and solid reagents. After 48 h the reactor was cooled. The Teflon liner was removed from the housing and centrifuged to separate the solid reaction products from the liquid. The liquid was decanted and distilled water (5 mL) was added to the solid. The solid was crushed and agitated to dissolve soluble salts. The mixture was centrifuged and the liquid decanted. This washing was done twice. The wet solid was dried in air at 120° C. for 12 h. The solid material was then transferred to a vial, crushed, and calcined. The solid was then ground, pressed, and sieved to a particle size range of 145 to 355 μm and tested for catalytic performance. This material had the nominal composition Mo₁V_{0.3}Sb_{0.2}Nb_{0.06}Ge_{0.3}Oₙ. A 0.15 g portion of the catalyst was diluted with SiC and loaded in a 4 mm ID reactor tube. Under propane ammoxidation conditions, 420° C., WHSV=0.15, and feed ratio C₃H₈/NH₃/O₂/He=1/1.2/3/12 an acrylonitrile yield of 54% was achieved at a propane conversion of 87% and an acrylonitrile selectivity of 62%.

### EXAMPLE #22

### Mo₁V_{0.2}Sb_{0.26}Nb_{0.09}Ti_{0.1}Nd_{0.05}Oₙ

To a 23 mL Teflon acid digestion bomb was added MoO₃ (1.20 g), Sb₂O₃ (0.316 g), and V₂O₅ (0.152 g). To the solid mixture was added 2 mL water. The suspension was magnetically stirred for 5 min. To the stirred mixture was added TiO₂ (0.833 mL of an aqueous slurry containing 0.08 g TiO₂ per mL) and Nd(OAc)₃ (0.417 mL of a 0.1M soln.). After another 5 min, a solution of ammonium niobium oxalate (1.50 mL, Nb=0.5M) was added and enough distilled water was added to obtain an 80% fill volume in the reactor. The acid digestion bomb was sealed and placed in an oven preheated to 175° C. and was continuously rotated to affect mixing of the liquid and solid reagents. After 48 h the reactor was cooled. The Teflon liner was removed from the housing and the slurry was transferred to a round bottom flask. The liquid was removed using a rotary evaporator. The solid was further dried in air at 120° C. for 12 h. The solid material was then transferred to a vial, crushed, and calcined for 2 h at 600° C. under N₂. The solid was then ground, pressed, and sieved to a particle size range of 145 to 355 μm and tested for catalytic performance. This material had the nominal composition Mo₁V_{0.2}Sb_{0.26}Nb_{0.09}Ti_{0.1}Nd_{0.005}Oₙ.

A 0.15 g portion of the catalyst was diluted with SiC and loaded in a 4 mm ID reactor tube. Under propane ammoxidation conditions, 430° C., WHSV=0.2, and feed ratio C₃H₈/NH₃/O₂/He=1/1.2/3/12 an acrylonitrile yield of 54% was achieved at a propane conversion of 89% and a acrylonitrile selectivity of 61%.

### EXAMPLE #23

### Mo₁V_{0.275}Sb_{0.2}Nb_{0.06}Ti_{0.1}Nd_{0.005}Oₙ

To a 125 mL Teflon acid digestion bomb was added MoO₃ (8.00 g), Sb₂O₃ (1.62 g), TiO₂ (0.444 g), V₂O₅ (1.390 g), and Nd(OAc)₃ (0.0893 g). Water (10 mL) was added and the mixture was magnetically stirred for 5 minutes. To the stirred mixture was added aqueous oxalic acid (11.12 mL, 0.5 M), and a Nb oxalate solution (8.30 mL, Nb=0.402M, oxalate/Nb 3). Enough distilled water was then added to obtain an 80% fill volume in the reactor. The acid digestion bomb was sealed and placed in an oven preheated to 175° C. and was continuously rotated to affect mixing of the liquid and solid reagents. After 64 h the reactor was cooled. The Teflon liner was removed from the housing. The product slurry was transferred to a round bottom flask and the solvent was removed using a rotary evaporator. The solid was further dried in air at 120° C. for 12 h. The solid material was then crushed and calcined under N₂ for 2 h at 600° C. The solid was then ground, pressed, and sieved to a particle size range of 145 to 355 μm and tested for catalytic performance. This material had the nominal composition Mo₁V_{0.275}Sb_{0.2}Nb_{0.06}Ti_{0.1}Nd_{0.05}Oₙ.

A 0.15 g portion of the catalyst was diluted with SiC and loaded in a 4 mm ID glass lined reactor tube. Under propane ammoxidation conditions, 420° C., WHSV=0.20, and feed ratio C₃H₈/NH₃/O₂/He=1/1.2/3/12 an acrylonitrile yield of 51% was obtained.

### EXAMPLE #24

### Mo₁V_{0.2}Sb_{0.225}Nb_{0.06}Ti_{0.1}Nd_{0.005}Oₙ

To a 125 mL Teflon acid digestion bomb was added MoO₃ (8.00 g), Sb₂O₃ (1.823 g), TiO₂ (0.444 g), V₂O₅ (1.011 g), and Nd(OAc)₃ (0.0893 g). Water (10 mL) was added and the mixture was magnetically stirred for 5 minutes. To the stirred mixture was added aqueous oxalic acid (11.12 mL, 0.5 M), and an ammonium Nb oxalate solution (6.67 mL, Nb=0.5M). Enough distilled water was then added to obtain an 80% fill volume in the reactor. The acid digestion bomb was sealed and placed in an oven preheated to 175° C. and was continuously rotated to affect mixing of the liquid and solid reagents. After 70 h the reactor was cooled. The Teflon liner was removed from the housing. The product slurry was transferred to a round bottom flask and the solvent was removed using a rotary evaporator. The solid was further dried in air at 120° C. for 12 h. The solid material was then crushed and calcined under N₂ for 2 hours at 600° C. The solid was then ground, pressed, and sieved to a particle size range of 145 to 355 μm and tested for catalytic performance. This material had the nominal composition Mo₁V_{0.2}Sb_{0.225}Nb_{0.06}Ti_{0.1}Nd_{0.005}Oₙ.

A 0.15 g portion of the catalyst was diluted with SiC and loaded in a 4 mm ID glass lined reactor tube. Under propane ammoxidation conditions, 420° C., WHSV=0.125, and feed ratio C₃H₈/NH₃/O₂/He=1/1.2/3/12 an acrylonitrile yield of 52% was obtained.

### EXAMPLE #25

### Mo₁V_{0.2}Sb_{0.225}Nb_{0.06}Ti_{0.1}Nd_{0.05}Oₙ

To a 125 mL Teflon acid digestion bomb was added MoO₃ (8.00 g), Sb₂O₃ (1.823 g), TiO₂ (0.444 g), V₂O₅ (1.011 g), and Nd(OAc)₃ (0.0593 g). Water (10 mL) was added and the mixture was magnetically stirred for 5 minutes. To the stirred mixture was added aqueous oxalic acid (1.12 mL, 0.5 M), and an ammonium Nb oxalate solution (6.67 mL, Nb=0.5M). Enough distilled water was then added to obtain an 80% fill volume in the reactor. The acid digestion bomb was sealed and placed in an oven preheated to 175° C. and was continuously rotated to affect mixing of the liquid and solid reagents. After 48 h the reactor was cooled. The Teflon liner was removed from the housing. The product slurry was spray dried. The resulting powder was further dried in air at 120° C. for 12 hours and calcined under N₂ for 2 h at 600° C. The solid was then ground, pressed, and sieved to a particle size range of 145 to 355 μm and tested for catalytic performance. This material had the nominal composition Mo₁V_{0.2}Sb_{0.225}Nb_{0.06}Ti_{0.1}Nd_{0.005}Oₙ.

A 0.15 g portion of the catalyst was diluted with SiC and loaded in a 4 mm ID glass lined reactor tube. Under propane ammoxidation conditions, 420° C., WHSV=0.15, and feed ratio C₃H₈/NH₃/O₂/He=1/1.2/3/12 an acrylonitrile yield of 50% was obtained.

### EXAMPLE #26

### Mo₁V_{0.2}Sb_{0.2}Nb_{0.04}Ti_{0.1}Nd_{0.005}Oₙ

To a 125 mL Teflon acid digestion bomb was added MoO₃ (8.00 g), Sb₂O₃ (1.6209). TiO₂ (0.444 g), V₂O₅ (1.011 g), and Nd(OAc)₃ (0.0893 g). Water (10 mL) was added and the mixture was magnetically stirred for 5 minutes. To the stirred mixture was added aqueous oxalic acid (5.56 mL, 0.5 M), and a Nb oxalate solution (5.53 mL, Nb=0.402M, oxalate/Nb=3). Enough distilled water was then added to obtain an 80% fill volume in the reactor. The acid digestion bomb was sealed and placed in an oven preheated to 175° C. and was continuously rotated to affect mixing of the liquid and solid reagents. After 48 h the reactor was cooled. The Teflon liner was removed from the housing. The product slurry was transferred to a round bottom flask and the solvent was removed using a rotary evaporator. The solid was further dried in air at 120° C. for 12 h. The solid material was then crushed and calcined under N₂ for 2 h at 600° C. The solid was then ground, pressed, and sieved to a particle size range of 145 to 355 μm and tested for catalytic performance. This material had the nominal composition Mo₁V_{0.2}Sb_{0.2}Nb_{0.4}Ti_{0.1}Nd_{0.005}Oₙ.

A 0.15 g portion of the catalyst was diluted with SiC and loaded in a 4 mm ID glass lined reactor tube. Under propane ammoxidation conditions, 420° C., WHSW=0.15, and feed ratio C₃H₈/NH₃/O₂/He=1/1.2/3/12 an acrylonitrile yield of 50% was obtained.

### EXAMPLE #27

### Mo₁V_{0.2}Sb_{0.225}Nb_{0.06}Ti_{0.1}Nd_{0.005}Oₙ

A 23 mL Teflon reactor liner was loaded with MoO₃ (1.0 g), V₂O₅ (1.487 mL of a 0.47 M solution)), Sb₂O₃ (3.177 mL of a 0.246 M slurry), TiO₂ (2.481 mL of a 0.280 M slurry), Nd(OAc)₃ (0.877 mL of a 0.04 M solution), ammonium niobium oxalate (1.042 mL of a 0.4 M solution having oxalate/Nb=2), oxalic acid (1.389 mL of a 0.5 M solution) and water (2.84 mL). The liner was placed in an acid digestion bomb, the bomb was then sealed and placed in an oven which was heated to 175° C. The bomb was continuously rotated to affect mixing of the liquid and solid reagents for a period of 48 hrs. The bomb was then cooled to room temperature, the teflon liner removed from the housing and the slurry was transferred to a round bottom flask. The liquid was removed using a rotary evaporator. The solid was further dried in air at 120° C. for 12 h. The solid material was then transferred to a vial, crushed, and calcined for 2 h at 600° C. under N₂. The solid was then ground, pressed, and sieved to a particle size range of 145 to 355 μm and tested for catalytic performance for the heterogeneous ammoxidation of propane to acrylonitrile. This material has the nominal composition Mo₁V_{0.2}Sb_{0.225}Nb_{0.06}Ti_{0.1}Nd_{0.005}Oₙ. At 420° C., WHSV=0.1 and a feed ratio of C₃H₈/NH₃/O₂/He=1/14/3/12, an acrylonitrile yield of 45% was obtained (97% propane conversion, 56% acrylonitrile selectivity). At 420° C., WHSV=0.2 and a feed ratio of C₃H₈/NH₃/O₂/He=1/14/3/12, an acrylonitrile yield of 54% was obtained (85% propane conversion, 64% acrylonitrile selectivity.

### EXAMPLE #28

### Corrosion Testing

Three catalysts having a nominal composition of Mo_{1.0}V_{0.3}Sb_{0.76}Nb_{0.06}Ti_{0.1}Nd_{0.005}Oₙ were prepared. One using the method similar to that described in Example 3 (VOSO₄ as the vanadium source) and the other two using a method similar to that described in Example 22 (V₂O₅ as the vanadium source). In each preparation, a small coupon of the 316 stainless steel metal was placed in the reactor with the slurry, left in the reactor during the reaction, and then removed at the end, washed, dried and weighed. The percent weight loss was then measured and the results for the tests are listed in Table 1 below:

TABLE 1

| | Vanadium | Coupon | Percent |
|---|---|---|---|
| Composition | Source | Material | Weight Loss |
| Mo_{1.0}V_{0.3}Sb_{0.176}Nb_{0.06}Ti_{0.1}Nd_{0.005}^{O}ₙ | VOSO₄ | 316 SS | 0.25 |
| Mo_{1.0}V_{0.3}Sb_{0.176}Nb_{0.06}Ti_{0.1}Nd_{0.005}^{O}ₙ | V₂O₅ | 316 SS | 0.08 |
| Mo_{1.0}V_{0.3}Sb_{0.176}Nb_{0.06}Ti_{0.1}Nd_{0.005}^{O}ₙ | V₂O₅ | 316 SS | 0.10 |

### The above data illustrates that the corrosiveness of the V₂O₅ is approximately one-half that of the sulfate preparation.

While the foregoing description and the above embodiments are typical for the practice of the instant invention, it is evident that many alternatives, modifications, and variations will be apparent to those skilled in the art in light of this description. Accordingly, it is intended that all such alternatives, modifications and variations are embraced by and fall within the spirit and broad scope of the appended claims.

We claim:

1. A catalyst composition comprising a mixed oxide of empirical formula:
Mo₁VₐSb_{b}Nb_{c}X_{d}LₑOₙ
wherein X is selected from the group consisting of Ti, Sn, Zr, Hf, and mixtures thereof;
L is selected from the group consisting of La, Pr, Nd, Sm, Eu, Gd, Th, Dy, Ho, Er, Tm, Yb, Lu and mixtures thereof;
0.1<a<0.8,
0.01<b<0.6,
0.01<c<0.2,
0.005<d<0.6,
0<e<0.04; and
n is number of oxygen atoms required to satisfy valance requirements of all other elements present in the mixed oxide with the proviso that one or more of the other elements in the mixed oxide can be present in an oxidation state lower than its highest oxidation state, and
a, b, c, d and e represent the molar ratio of the corresponding element to one mole of Mo..

2. The catalyst composition of claim 1, wherein 0.2<a<0.4, 0.1<b<0.3, 0.04<c<0.1, 0.01<d<0.2, and 0.001<e<0.016.

3. The catalyst composition of claim 1 wherein X is selected from the group consisting of elements Ti, Sn and mixtures thereof.

4. The catalyst composition of claim 1 wherein L is selected from the group consisting of elements Nd, Pr and mixtures thereof.

5. The catalyst composition of claim 1, wherein the catalyst composition a support selected from the group consisting of silica, alumina, zirconia, titania, or mixtures thereof.

6. The catalyst composition of claim 11, wherein the support comprises about 10 to about 70 weight percent of the catalyst.

7. A process for the ammoxidation of a saturated or unsaturated or mixture of saturated and unsaturated hydrocarbon to produce an unsaturated nitrile, said process comprising contacting the saturated or unsaturated or mixture of saturated and unsaturated hydrocarbon with ammonia and an oxygen-containing gas in the presence of a catalyst composition comprising a mixed oxide of empirical formula:
Mo₁VₐSb_{b}Nb_{c}X_{d}LₑOₙ
wherein X is selected from the group consisting of Ti, Sn, Zr, Hf, and mixtures thereof;
L is selected from the group consisting of La, Pr, Nd, Sm, Eu, Gd, Th, Dy, Ho, Er, Tm, Yb, Lu and mixtures thereof;
0.1<a<0.8,
0.01<b<0.6,
0.01<c<0.2,
0.005<d<0.6,
0.005<e<0.04; and
n is number of oxygen atoms required to satisfy valance requirements of all other elements present in the mixed oxide with the proviso that one or more of the other elements in the mixed oxide can be present in an oxidation state lower than its highest oxidation state, wherein a, b, c, d and e represent the molar ratio of the corresponding element to one mole of Mo.

8. The process of claim 17, wherein in the catalyst composition: 0.2<a<0.4, 0.1<b<0.3, 0.04<c<0.1, 0.01<d<0.2, and 0.001<e<0.016.

9. The process of claim 7, wherein X is selected from the group consisting of Ti, Sn and mixtures thereof.

10. The process of claim 7, wherein L is selected from the group consisting of Nd, Pr and mixtures thereof.

11. The process of claim 7, wherein the catalyst composition comprises a support selected from the group consisting of silica, alumina, zirconia, titania, or mixtures thereof.

12. The process of claim 11, wherein the support comprises about 10 to about 70 weight percent of the catalyst.

13. A process for preparing a mixed metal oxide catalyst comprising molybdenum, vanadium, antimony, niobium, at least one element select from the group consisting of titanium, tin, germanium, zirconium, and hafnium and at least one lanthanide selected from the group consisting of lanthanum, cerium, praseodymium, neodymium, samarium, europium, gadolinium, terbium, dysprosium, holmium, erbium, thulium, ytterbium and lutetium, wherein the process comprising mixing and reacting source compounds of the metal components and wherein the vanadium source is vanadium oxide (V₂O₅);

14. The process of claim 13, wherein (i) the niobium source is niobic acid, (ii) the niobic acid is combined with source compounds of the metal components in the presence of oxalic acid (H₂O₂CCO₂H), and (iii) the oxalic acid to niobium molar ratio is at least 4.5 to 1.

15. A process for preparing a mixed metal oxide catalyst comprising molybdenum, vanadium, antimony, niobium, at least one element select from the group consisting of titanium, tin, germanium, zirconium, and hafnium and at least one lanthanide selected from the group consisting of lanthanum, cerium, praseodymium, neodymium, samarium, europium, gadolinium, terbium, dysprosium, holmium, erbium, thulium, ytterbium and lutetium, the process comprises comprising the following steps:
(i) preparing a first mixture comprising source compounds of Mo, V, Sb at least one of Ti, Sn, Ge, Zr, or Hf, and at least one of at least one of La, Ce, Pr, Nd, Sm, Eu, Gd, Th, Dy, Ho, Er, Tm, Yb, Lu, wherein the V source compound is vanadium oxide (V₂O₅);
(ii) preparing a second mixture comprising a niobium source compound and oxalic acid (H₂O₂CCO₂H) wherein the oxalic acid to niobium molar ratio is at least 4.5 to 1;
(iii) admixing, in a reaction vessel said first mixture and said second mixture in an aqueous solvent to form a reaction medium;
(iv) optionally adding additional aqueous solvent to the reaction vessel;
(v) sealing the reaction vessel;
(vi) reacting the reaction medium at a temperature greater than 100° C. and a pressure greater than ambient pressure for a time sufficient to form a mixed metal oxide;
(vii) optionally cooling the reaction medium; and
(viii) drying and calcining the mixed metal oxide.

16. The process of claim is, wherein the niobium source compound is niobic acid (Nb₂O₅.nH₂O).
